(19) United States
(12) Patent Application Publication
(10) Pub. No.: US 2012/0148712 A1
(43) Pub. Date: Jun. 14, 2012

(54) PHYLUM ARTHROPODA BASED NUTRITIONAL SUPPLEMENT

(76) Inventors: Dianne Lee Guilfoyle, Redlands, CA (US); Cameron David Noerr, Colton, CA (US)

(21) Appl. No.: 13/300,252

(22) Filed: Nov. 18, 2011

Related U.S. Application Data
(60) Provisional application No. 61/422,987, filed on Dec. 14, 2010.

(51) Int. Cl. A23L 1/305; G01N 33/02

(52) U.S. Cl. 426/231; 426/444; 426/648; 426/617

(57) ABSTRACT

An arthropod based nutritional supplement may comprise one species within the arthropod phylum or two or more species within the arthropoda phylum combined together. These arthropods are powderized and its nutritional profile analyzed. Based on the nutritional profile of the powderized arthropods, additional powderized arthropoda or additional nutritional components may be added in order to develop a nutritional profile that can satisfy a particular need for a body builder, a man, a woman or provide generic meal replacement.

### CROSS-REFERENCE TO RELATED APPLICATIONS

This application claims priority to U.S. Prov. Pat. App. No. 61/422,987, filed on Dec. 14, 2010, the entire contents which are expressly incorporated herein by reference.

### STATEMENT RE: FEDERALLY SPONSORED RESEARCH/DEVELOPMENT

Not Applicable

### BACKGROUND

The present invention relates to an arthropod based nutritional supplement.

The elderly, body builders, athletes, men, women and children, etc. all have nutritional needs that may not be satisfied by their particular diet. For example, vegetarians may need to supplement their food intake with additional nutritional supplements to make up for those nutritional components not provided by the vegetarian diet. Body builders may need additional nutritional components to achieve their goals. Women have different nutritional needs compared to men. In response, traditional nutritional supplements have been developed to cater to the particular needs of individuals and groups of people. However, these nutritional supplements are usually processed with whey and soy as the primary base ingredient and may not provide optimal performance for individuals who may be allergic to soy or are seeking an alternate source.

Moreover, although some manufacturers may sell canned insects, dried insects or insect powder, these products are used mainly for exotic culinary or novelty candy purposes. They are not designated for use as a nutritional supplement, meal replacement or survival purposes.

Accordingly, there is a need in the art for an improved nutritional supplement.

### BRIEF SUMMARY

The nutritional supplement disclosed herein addresses the needs discussed above, discussed below and those that are known in the art.

The nutritional supplement is based on arthropods. In particular, the arthropods is cooked and dehydrated and the dehydrated arthropods are analyzed for its nutritional profile, including but not limited to its fat, protein, carbohydrate profile, as well as its amino acid profile. The arthropods may also be tested for metals, pesticides and aerobic plate counts which is a Hazard Analysis and Critical Control Point (HACCP). Based on the nutritional profile of the dehydrated arthropods, additional nutritional components are added to the dehydrated arthropods to satisfy a particular purpose. By way of example and not limitation, additional nutritional components may be added to the powderized arthropods to develop a nutritional supplement for women, men, body building, meal replacement, etc. Although the arthropod based nutritional supplement disclosed herein is discussed in relation to a cricket, any one or a combination of species of arthropoda phylum may be used to develop the arthropod base nutritional supplement. It is also contemplated that no additional nutritional components are added to the base powderized arthropods. Rather, two or more different species of the arthropoda phylum may be processed and mixed together to formulate a complete arthropod base nutritional supplement.

More particularly, a method of manufacturing an arthropod based nutritional supplement is disclosed. The method may comprise the steps of separating arthropods from frass; freezing the arthropods; cooking and pasteurizing the arthropods in boiling liquid to kill germs, viruses and parasites while wrapped in a liquid permeable layer; removing the arthropods from the boiling liquid; pre-treating the arthropods in a brine to destroy pathogens that may cause food borne illnesses; mechanically removing liquid from the liquid permeable layer and the arthropods; dehydrating the arthorpods; powderizing the dehydrated arthropods to a powder form; and mixing powdered arthropods with nutritional supplements to achieve a desired amino acid profile based on a nutritional profile of the powdered arthropoda. Each step of the method for fabricating the arthropod based nutritional supplement formulation must be accomplished by meticulous guidelines comprised of appropriate HACCP approved process controls and strict adherence to good manufacturing practices.

The separating step may include the step of placing a funnel in a container with the arthropods stored therein so that the arthropods can climb onto an inner surface of the funnel. The funnel may have a cylindrical shape.

The freezing step may include the step of placing the arthropoda into a freezer maintained at a temperature at around or below zero degrees Fahrenheit.

The cooking step may include the step of wrapping the arthropoda in cheese cloth and dunking the wrapped arthropods in the boiling liquid.

The pre-treating step may include the step of soaking the arthropods in the brine for about five minutes.

The removing liquid step may include the step of wringing out the liquid from the liquid permeable layer.

The dehydrating step may include the step of placing the arthropods within a dehydrator until the arthropods are dehydrated.

The dehydrating step may include the step of placing the arthropods within an oven to dry the arthropods and stirring the arthropods while being dried in the oven.

The mixing step may include the step of adding additional nutritional components based on the nutritional profile of the powderized arthropods.

The powderizing step may be accomplished with a food processor or blender.

Additionally, a method of processing an arthropod for human consumption is disclosed. The method may comprise the steps of analyzing a nutritional profile of an arthropod; processing the arthropod for human consumption; and mixing whey protein isolate and coconut to the processed arthropod to optimize the nutritional profile of the mixture for human consumption based on the analyzed nutritional profile of the arthropod.

The analyzing step may comprise the step of analyzing a macro nutritional profile of the arthropod. Alternatively or additionally, the analyzing step may comprise the step of analyzing a micro nutritional profile of the arthropod.

The arthropod may be one or a combination of a cricket, mealworm, fly, ant and locust.

The arthropod may be a mealworm. The processed mealworm may be mixed with whey protein isolate and coconut in the following weight percentages: processed mealworm 35% to 55%, whey protein isolate 20% to 40% and coconut 15% to 35%.

The arthropod may be a cricket. The processed cricket may be mixed with whey protein isolate and coconut in the following weight percentages: processed cricket 40% to 60%, whey protein isolate 15% to 35% and coconut 15% to 35%.

The arthropod may be a fly. The processed flies may be mixed with whey protein isolate and coconut in the following weight percentages: processed flies 30% to 50%, whey protein isolate 25% to 45% and coconut 15% to 35%.

The arthropod may be an ant. The processed ants may be mixed with whey protein isolate and coconut in the following weight percentages: processed ant 40% to 60%, whey protein isolate 10% to 30% and coconut 20% to 40%.

The arthropod may be a locust. The processed locusts may be mixed with whey protein isolate and coconut in the following weight percentages: processed locusts 40% to 60%, whey protein isolate 10% to 30% and coconut 20% to 40%.

Moreover, a method of processing an arthropod for human consumption is disclosed. The method may comprise the steps of analyzing an amino acid profile of an arthropod; processing the arthropod for human consumption; and mixing an additional nutritional component to the processed arthropod to optimize the amino acid profile of the mixture for human consumption based on the analyzed nutritional profile of the arthropod.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a container for storing arthropods and separating the arthropods from frass and dead arthropods;

FIG. 2 is a perspective view of a freezer for freezing the arthropods separated as shown in FIG. 1;

FIG. 3 illustrates a pot of boiling water for cooking arthropods;

FIG. 4 illustrates a pot of cold water to stop the cooking process shown in FIG. 3;

FIG. 5 illustrates a dehydrator for drying the arthropods;

FIG. 6 illustrates an oven for providing an alternate method for drying the arthropods; and

FIG. 7 is a perspective view of a blender for powderizing the arthropods.

### DETAILED DESCRIPTION

Referring now to the drawings, a nutritional supplement based upon one or more species within the arthropoda phylum is shown and also disclosed herein. The arthropod base nutritional supplement is derived in the following manner. The nutritional profile of processed arthropods (i.e., cooked and dehydrated) are initially analyzed. Based upon the nutritional profile of the processed arthropods, additional nutritional components may be combined with the processed arthropods. For example, different nutritional components may be added to the processed arthropods based on the purpose of the nutritional supplement such as meal replacement, muscle building, recovery, etc.

By way of example and not limitation, the following nutritional supplement example is discussed in relation to a cricket based nutritional supplement for the purposes of meal replacements. Initially, the cricket is processed into a powder form. The processed cricket is then analyzed for its nutritional profile including but not limited to its fat, carbohydrates, protein and amino acids profile. For example, the cricket powder may have 7.5 grams of fat, 5 grams of carbohydrates and 37.5 grams of protein for every 50 grams of cricket powder. Also, the cricket powder may have varying amounts of lycine, branch chain amino acids, tyrosine, phenylalanine, proline as well as palmatic acid, lignans and ecdysterone. Based on the nutritional profile of the processed cricket, additional nutritional components may be added to the cricket powder in order to achieve the designed purpose of the resultant nutritional supplement. If the nutritional supplement is to serve as a meal replacement, for every 50 grams of cricket powder, 5 grams of glutamine and 200 milligrams of vitamin C may be added. The purpose of the additional nutritional component is to bring the amino acid profile of the processed cricket into a balanced amino acid profile to create a more anabolic atmosphere. This allows the human body to be able to better assimilate the arthropod based nutritional supplement. To thoroughly combine the additional nutritional components with the cricket powder, the cricket powder and additional nutritional components may be mixed or blended in a food processor or blending machine in the proportions mentioned above. The food processor or blending machine mixes the cricket powder and the additional nutritional components which may be provided in a powder form to thoroughly mix the two together to achieve a uniform blend of macro and micro nutrients necessary for a complete nutritional supplement or meal replacement product. After mixing, the meal replacement or the arthropod based meal may be packaged and sold to the consuming public. Optional flavoring such as xylitol, ginger, cinnamon and peppermint may be added to the arthropod based nutritional supplement. The powder form of the arthropod based nutritional supplement may be mixed in with water and flavored liquid then consumed.

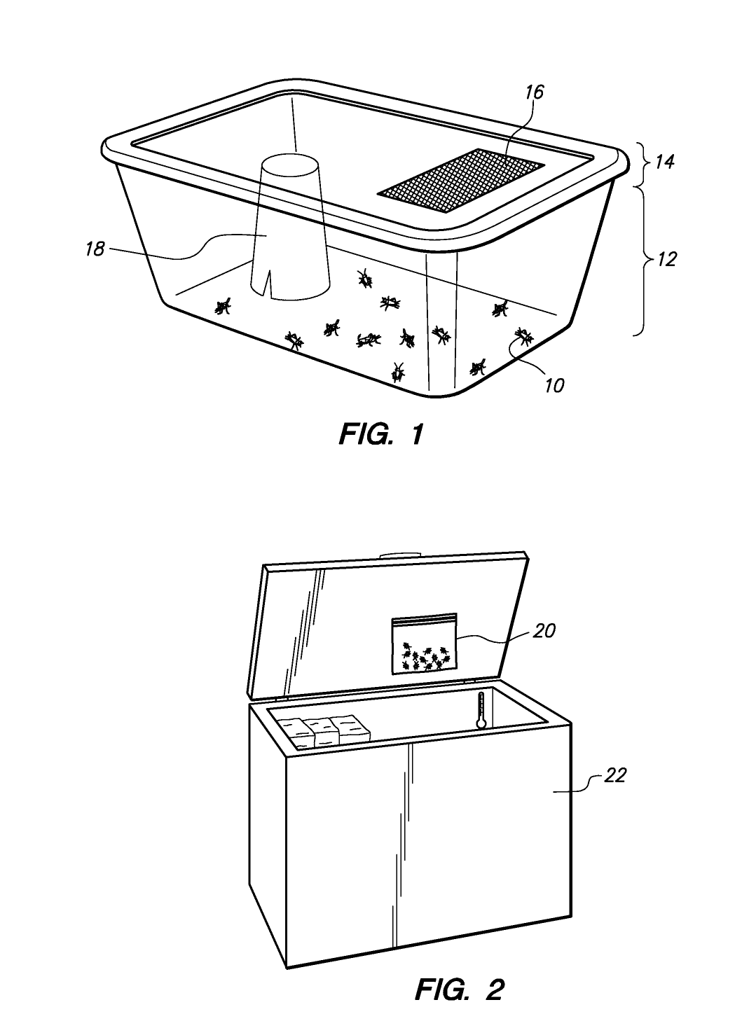

More particularly, referring now to FIG. 1, upon receipt of the arthropods 10, they 10 are inspected for quality and placed in a holding container 12. A lid 14 is placed on the container 12 to cover the container 12 to prevent escape of the arthropods. The lid 14 may have a hole covered with a mesh 16 to allow the arthropods 10 to breathe during storage. Typically, the guts of the arthropods will be empty of frass due to fasting during transport from the arthropod harvester. If the time for transport is not sufficient to empty the guts of the arthropods of frass, the arthropods 10 may be stored in the container 12 until the gut of the arthropods are empty. Once empty, the arthropods are removed from the container 12 and then frozen.

If the arthropods are not immediately processed upon arrival, the arthropods may be fed fresh kale or combined sources of fresh fruits and vegetables until processing of the arthropods. Fresh kale is washed to remove any pesticide and placed in the container 12. Condensation is not allowed to accumulate in the container 12. When the arthropods are ready for processing, the kale is removed and the arthropods are allowed to fast to expel the frass from their gut. Once the frass is removed from the guts of the arthropods, the arthropods are removed from the container 12.

To remove the arthropods 10 from the container 12, a hollow tube 18 (e.g., pyramid shape or cone shape) is placed in the container 12 after the guts of the arthropods are emptied. The arthropods climb up on the hollow tube 18 while the frass and dead arthropods remain at the bottom of the container 12. The hollow cylinder 18 with the arthropods 10 climbing up thereon is used to place the arthropods 10 in a plastic bag 20 to prepare the arthropods for freezing in freezer 22, as shown in FIG. 2. The plastic bag 20 is opened and the arthropods 10 on the hollow tube 18 are shaken off into the plastic bag 20. Once the arthropods 10 are in the plastic bag 20, the plastic bag 20 may be sealed and placed in the freezer 22. Typically, the freezer is maintained at below zero degrees Fahrenheit or minus 18 degrees Celsius. The arthropods are frozen and kept frozen until the time of cooking. The arthropods once frozen are not allowed to thaw or even partially thaw otherwise they are discarded.

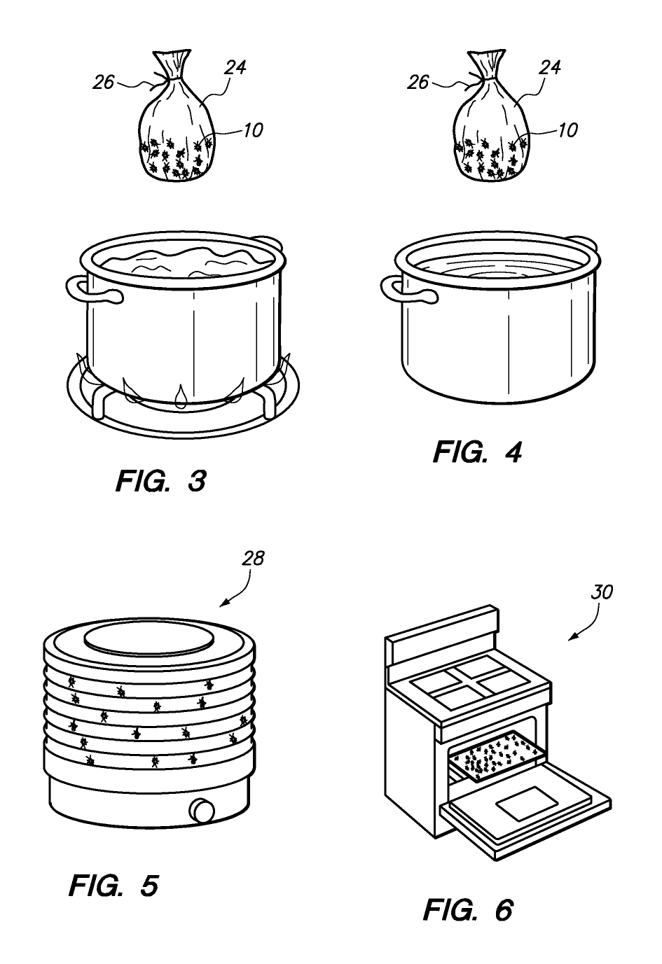

To process the arthropods, the following equipment is needed. An oven preheated to 200 degrees Fahrenheit, clean heat pans or pizza pans with vented bottoms, a large stainless steel kettle with lid with water which is brought to a rolling boil, a large pot of iced water and clean towels. The insects are removed from the freezer 22 and placed into a colander to be rinsed. Although this partially thaws the arthropods, the arthropods are cooked immediately usually within fifteen minutes. The arthropods are rinsed in the colander and wrapped in cheese cloth 24 and the top of the cheese cloth 24 is tied off with a string 26. The arthropods 10 remain in the cheese cloth 24 during cooking. The arthropods are dropped into the boiling water (see FIG. 3) which causes the water to stop boiling. As additional heat is introduced into the water, the water begins to boil again pasteurizing the arthropods. At this time, the arthropods are removed from the boiling water and placed into the iced cold water (see FIG. 4) for two minutes to stop the cooking process.

Prior to drying the arthropods, the arthropods are pre-treated to destroy pathogens that could cause food poisoning illnesses such as escherichia coli O157:H7, salmonella species, hysteria monocytogenes. The pre-treatment may consist of a preparation of brine consisting of either vinegar water (one and one quarter tablespoon of vinegar to every eight cups of water), salt water (one tablespoon salt to every eight cups of water), vitamin C water (one thousand milligrams vitamin C to two cups of water) or lemon water (one third cup lemon juice to every two cups of water). The arthropods are soaked in the brine for five minutes. After soaking the arthropods in the brine, a clean towel is wrapped about the cheese cloth and the water in the cheese cloth 24 and arthropods 10 are wrung to remove as much water as possible.

To thoroughly dry the arthropods, the arthropods 10 may be dried in a dehydrator 28 or a conventional oven 30. When using a dehydrator 28 (see FIG. 5), the dehydrator is set to 150 degrees Fahrenheit to 165 degrees Fahrenheit. Preferably, the dehydrator has multiple stacks or trays. The string 26 is removed so that the cheese cloth 24 can be laid flat. The cheese cloth is cut to the size of the tray in the dehydrator 28. With the arthropods 10 on the top side of the cheese cloth 24, the cheese cloth is laid on each tray of the dehydrator 28 to prevent the arthropods from falling through vents in the tray of the dehydrator 28. Once the arthropods 10 are dehydrated, the dehydrated arthropods are transferred into a bowl. Most of the arthropods 10 will simply fall into the bowl once they are dry. However, some components (e.g. antennas and legs) of the arthropods may stick to the cheese cloth 24. In this instance, optionally, one may pick off the legs and antennas and transfer them into the bowl.

When using a conventional oven 30 (see FIG. 6), the arthropods are removed from the cheese cloth 24 and then spread thinly and evenly across the surface of the pan to be placed in the oven 30. The oven temperature is set to about 200-250 degrees Fahrenheit. The arthropods are stirred during the drying process every 20 to 30 minutes with either a health department approved serving glove on your hands or with a spatula. To stir the insects in the oven by hand with a serving glove, the arthropods are removed from the oven 30 to a clean surface. The arthropods are picked up and moved and checked for dryness. To stir insects dried in the oven using a spatula, the insects are removed from the oven to a clean dry surface. One hand in placed near the edge of the pan with the spatula brought under the arthropods toward the opposing hand. The arthropods are then stirred and turned over. This process is repeated every 20 to 30 minutes until the arthropods are dry.

To check for dryness of the arthropods, one arthropod is removed from the oven or dehydrator and allowed to cool to room temperature. The arthropod is squeezed in your hand or between a piece of paper towel to check for moisture. If no moisture is present, then the arthropods are dry enough to proceed to the next step.

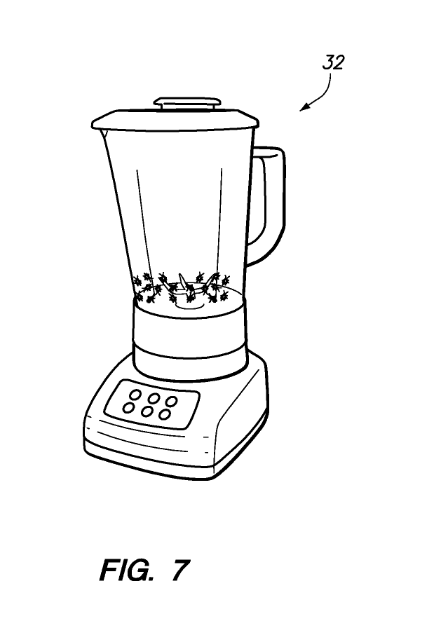

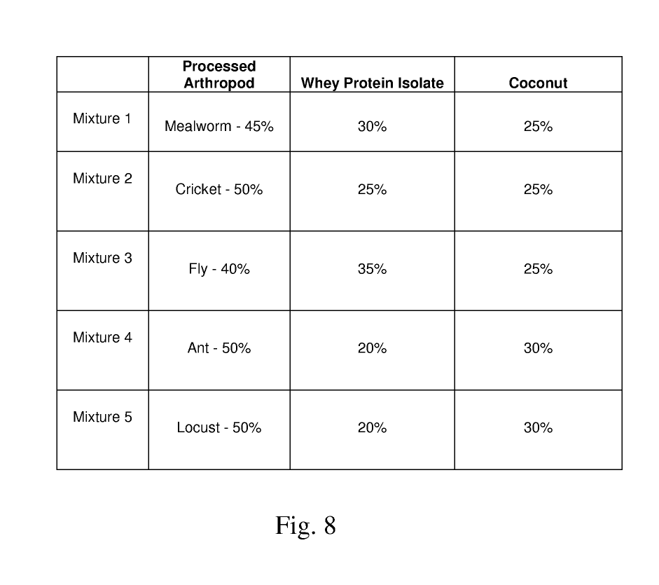

After dehydration or drying, the arthropods are blended in a food processor or blender 32 (see FIG. 7) until the arthropods are in a fine powder form.

The processed arthropods are analyzed for its fat, carbohydrate and protein profile as well as amino acid profile. Based on the nutritional profile of the powdered arthropods, additional nutritional components are added to the powdered arthropods. As discussed above, for the crickets, 50 grams of cricket powder contains 7.5 grams of fat, 5 grams of carbohydrates and 37.5 grams of protein. The cricket powder also has a nutritional profile that includes ecdysterone, lignans, palmitic acid, lycine, branch chain amino acids, tyrosine, phenylalanie and proline. Based on this nutritional profile of the cricket powder, 5 grams of glutamine and 200 milligrams of vitamin C may be added to the base cricket powder to make a nutritional supplement to serve as a meal replacement. Flavorings may be added such as xylitol, ginger, cinnamon and peppermint. The arthropod nutritional supplement is then packaged and provided to the public. The public may consume the powder nutritional supplement by mixing the powder nutritional supplement with water or juice or flavored liquid.

The processed arthropods have a particular amino acid profile. The amino acids profile of the arthropod may be sub-optimal in creating an anabolic atmosphere so that the nutrition found in the arthropod cannot be effectively absorbed into the human body. The added nutritional components (e.g., glutamine for crickets) is added to bring/adjust the amino acid profile of the processed arthropods so as to have a more balanced amino acid profile so that the arthropod based nutritional supplement is more readily assimilated into the human body.

Other nutritional components (e.g., Vitamin C) can be added to the processed arthropods to take advantage of various excesses found in the amino acid profile of the arthropods. By way of example and not limitation, vitamin c can be added to the processed cricket to take advantage of the high levels of lysine and proline inherent in the processed cricket.

Although the above discussion of the arthropod based nutritional supplement was discussed in relation to a cricket based nutritional supplement, other species within the arthropoda phylum are also contemplated such as locust, grasshopper, beetles, ants, bees, grubs and any other invertebrate having jointed limbs and a segmented body with an exoskeleton made of chitin, also spiders, scorpions and all other animals within the Phylum Arthropoda. The arthropod based nutritional supplement may be based on any one or a combination of species within arthropoda phylum discussed herein or generally in the phylum arthropoda. It is also contemplated two or more different species within the arthropoda phylum may be combined and processed to provide for a balanced amino acid profile without the additional of additional nutritional components.

It is also contemplated that the arthropod based nutritional supplement may be packaged to provide a meal ration for the United States military to be carried and consumed by our soldiers for the purposes of restoration of strength and energy when commonly available food sources are scarce or unavailable or even as the primary source of energy.

In a different embodiment, the macro and micro nutrient profiles of the processed arthropods may be analyzed. One or more additional nutritional components (e.g., whey protein isolate and coconut) may be mixed in with the processed arthropods to bring the macro and micro nutrient profiles of the combination of the mixed processed arthropods and one or more nutritional components to an optimal level so that the human body or other living mammal (e.g., dog, cat, and animals) may more efficiently absorb (i.e., extract) the nutrients and assimilate (i.e., utilize) the absorbed nutrients more efficiently for a specific purpose such as muscle building, meal replacement, sleep aid or fat burn.

By way of example and not limitation, the processed crickets may be mixed with whey protein isolate and coconut in the weight percentages shown in FIG. 8. The weight percentages of each component (e.g., arthropod, whey protein isolate and coconut) may be increased and decreased plus or minus 10% to design the mixture of the processed arthropods, whey protein isolate and coconut for a specific purpose such as sleep aid, muscle building, meal replacement or fat burn. Other purposes are also contemplated.

Additionally, the cricket is exemplary and does not limit application of the various aspects disclosed herein to other arthropods. Other arthropods processed by the method discussed herein may be mixed with whey protein isolate and coconut. By way of example and not limitation, if a mealworm is used, the processed mealworm may be analyzed for its macro and micro nutritional profile. After the analysis, whey protein isolate and coconut may be mixed with the processed mealworm to bring the macro and micro nutritional profile to an optimal level for absorption and assimilation in the weight percentages shown in the table of FIG. 8. The weight percentages of the processed mealworm, whey protein isolate and coconut may be further increased or decreased by 10% to design the mixture for a specific purposes such as muscle building, meal replacement, fat burn, sleep aid as well as other uses. The same technique may be applied to other types of arthropods (e.g., fly, ant, locust, or other arthropod).

The mixture of the processed arthropod, whey protein isolate and coconut may also be considered a nutritional base upon which selected nutrients may be added to further enhance its nutritional properties to increase absorption, assimilation and/or further a purpose (e.g., sleep aid, fat burn, meal replacement, muscle building). The mixture may also be provided in the form of a powder, bar or liquid to consumers.

The processed arthropods may be retrieved in the form of but not limited to whole dried insects, and/or dried powder from sources such as but not limited to manufacturing plants, and/or insect farmhouses and/or personally grown and/or harvested insects.

The above description is given by way of example, and not limitation. Given the above disclosure, one skilled in the art could devise variations that are within the scope and spirit of the invention disclosed herein, including various ways of separating the arthropods from the frass and other dead arthropods. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of manufacturing an arthropod based nutritional supplement, the method comprising the steps of:
separating arthropods from frass;
freezing the arthropods;
cooking the arthropods in boiling liquid while wrapped in a liquid permeable layer;
removing the arthropods from the boiling liquid;
pre-treating the arthropods in a brine to destroy pathogens that may cause food borne illnesses;
mechanically removing liquid from the liquid permeable layer and the arthropods;
dehydrating the arthropods;
powderizing the dehydrated arthropods to a powder form;
mixing powdered arthropods and nutritional supplements to achieve a desired amino acid profile based on a nutritional profile of the powdered arthropods.

2. The method of claim 1 wherein the separating step including the step of placing a funnel in a container with the arthropods stored therein so that the arthropods can climb onto an inner surface of the funnel.

3. The method of claim 2 wherein the funnel has a cylindrical shape.

4. The method of claim 1 wherein the freezing step includes the step of placing the arthropods into a freezer maintained at a temperature at around or below zero degrees Fahrenheit.

5. The method of claim 1 wherein the cooking step includes the step of wrapping the arthropods in cheese cloth and dunking the wrapped arthropods in the boiling liquid.

6. The method of claim 1 wherein the pre-treating step includes the step of soaking the arthropods in the brine for about five minutes.

7. The method of claim 1 wherein the removing liquid step includes the step of wringing out the liquid from the liquid permeable layer.

8. The method of claim 1 wherein the dehydrating step includes the step of placing the arthropods within a dehydrator until the arthropods are dehydrated.

9. The method of claim 1 wherein the dehydrating step includes the step of placing the arthropods within an oven to dry the arthropods and stirring the arthropods while being dried in the oven.

10. The method of claim 1 wherein determination of the nutritional profile of the processed arthropods are accomplished by way of a laboratory analysis.

11. The method of claim 1 wherein the mixing step includes the step of adding additional nutritional components based on the nutritional profile of the powder arthropods.

12. The method of claim 1 wherein the powderizing step is accomplished with a food processor or blender.

13. The method of claim 1 further comprising the step of analyzing the arthropods mixed with the nutritional supplements to determine the nutritional profile of the mixed arthropoda and the nutritional supplements.

14. A method of processing an arthropod for human consumption, the method comprising the steps of:
analyzing a nutritional profile of an arthropod;
processing the arthropod for human consumption;
mixing whey protein isolate and coconut to the processed arthropod to optimize the nutritional profile of the mixture for human consumption based on the analyzed nutritional profile of the arthropod.

15. The method of claim 14 wherein the analyzing step comprises the step of analyzing a macro nutritional profile of the arthropod.

16. The method of claim 14 wherein the analyzing step comprises the step of analyzing a micro nutritional profile of the arthropod.

17. The method of claim 14 wherein the arthropod is one or a combination of a cricket, mealworm, fly, ant and locust.

18. The method of claim 14 wherein the arthropod is a mealworm and the processed mealworm is mixed with whey protein isolate and coconut in the following weight percentages: processed mealworm 35% to 55%, whey protein isolate 20% to 40% and coconut 15% to 35%.

19. The method of claim 14 wherein the arthropod is a cricket and the processed cricket is mixed with whey protein isolate and coconut in the following weight percentages: processed cricket 40% to 60%, whey protein isolate 15% to 35% and coconut 15% to 35%.

20. The method of claim 14 wherein the arthropod is a fly and the processed flies are mixed with whey protein isolate and coconut in the following weight percentages: processed flies 30% to 50%, whey protein isolate 25% to 45% and coconut 15% to 35%.

21. The method of claim 14 wherein the arthropod is an ant and the processed ants are mixed with whey protein isolate and coconut in the following weight percentages: processed ant 40% to 60%, whey protein isolate 10% to 30% and coconut 20% to 40%.

22. The method of claim 14 wherein the arthropod is a locust and the processed locusts are mixed with whey protein isolate and coconut in the following weight percentages: processed locusts 40% to 60%, whey protein isolate 10% to 30% and coconut 20% to 40%.

23. A method of processing an arthropod for human consumption, the method comprising the steps of:
analyzing an amino acid profile of an arthropod;
processing the arthropod for human consumption;
mixing an additional nutritional component to the processed arthropod to optimize the amino acid profile of the mixture for human consumption based on the analyzed nutritional profile of the arthropod.
